(19) United States
(12) Patent Application Publication
(10) Pub. No.: US 2010/0312204 A1
(43) Pub. Date: Dec. 9, 2010

(54) Menstrual Cup Device and Method of Use Thereof

(76) Inventor: Miin-Tsang Sheu, Changhua City (TW)

Correspondence Address: Jackson Intellectual Property Group PLLC, 106 Starvale Lane, Shipman, VA, 22971, US

(21) Appl. No.: 12/479,773

(22) Filed: Jun. 6, 2009

(51) Int. Cl. A61F 5/451

(52) U.S. Cl. 604/330

(57) ABSTRACT

A menstrual cup device and a method of us thereof includes a menstrual cup made of expandable material so as to be compressed, the menstrual cup having resilience for restoring to its original shape after being compressed, the menstrual cup comprising a pulling stem at a lower end thereof, a circular lip at an open upper end thereof, and a circular groove below the circular lip; and a collection bag having a resilient restraining ring at an open upper end thereof; thereby, the resilient restraining ring being expanded to be constricted in the circular groove of the menstrual cup, the circular lip being adapted to prevent the resilient restraining ring from disengagement, the menstrual cup with the collection bag being compressed and inserted into the vagina for collecting menstrual blood, the collection bag being disposable and replaceable.

### BACKGROUND OF THE INVENTION

1. Field of the Invention

The present invention relates to a menstrual cup device and a method of use thereof, and more particularly to one having a menstrual cup and a collection bag, which is convenient for replacement and is safe and hygienic in use.

2. Description of the Prior Art

In recent years, there has been a renewed interest in the use of the menstrual cup instead of the napkin and the tampon. The menstrual cup has a soft cup for receiving menstrual blood. It is re-useable and convenient.

Conventional menstrual cups, such as U.S. Pat. Nos. 891,761, 1,996,242, 2,089,113, 5,827,248, 323,212, use a soft cup to be inserted into the vagina for receiving menstrual blood. Removal of the menstrual cup from the vagina should be accomplished on a regular basis for emptying, cleaning and hygienic purposes. This is inconvenient to the user. In addition, the user has to carry a spare menstrual cup and a package bag for the dirty menstrual cup in case it is not convenient for the user to clean the menstrual cup immediately. This may cause hygienic problems or blood leakage. Sometimes, the user is embarrassed by the problem to clean the menstrual cup at the sink of a public lavatory. This is a main reason that the menstrual cup is not generally used.

### SUMMARY OF THE INVENTION

According to a first aspect of the present invention, there is provided a menstrual cup device, comprising:

a menstrual cup made of expandable material so as to be compressed, the menstrual cup having resilience for restoring to its original shape after being compressed, the menstrual cup comprising a pulling stem at a lower end thereof, a circular lip at an open upper end thereof, and a circular groove below the circular lip; and

a collection bag having a resilient restraining ring at an open upper end thereof;

thereby, the resilient restraining ring being expanded to be constricted in the circular groove of the menstrual cup, the circular lip being adapted to prevent the resilient restraining ring from disengagement, the menstrual cup with the collection bag being compressed and inserted into the vagina for collecting menstrual blood, the collection bag being disposable and replaceable.

According to a second aspect of the present invention, there is provided a method of use for applying a menstrual cup device, comprising the following steps of:
(a) expanding a resilient restraining ring of a collection bag to be located in a circular groove of a menstrual cup for lowering the risk of disengagement;
(b) providing a circular rib below the resilient restraining ring fitted in the circular groove;
(c) placing the collection bag in the menstrual cup to become an integral one;
(d) compressing the menstrual cup and the collection bag;
(e) inserting the compressed menstrual cup and the collection bag into the vagina, restoring the menstrual cup to its original shape with the collection bag wrapping a circular lip provided at an open upper end of the menstrual cup for abutting against the vaginal canal;
(f) removing the menstrual cup with the collection bag from the vagina for replacement, with the circular rib below the resilient restraining ring to prevent the resilient restraining ring form disengagement; and
(g) taking the collection bag out via the resilient restraining ring and replacing a new one.

Preferably, the menstrual cup further comprises a circular rib below the circular groove, the circular rib being formed with at least one gap for a user to take the collection bag out easily.

### BRIEF DESCRIPTION OF THE DRAWINGS

FIG. 1 is an exploded view of the present invention;

FIG. 1A is a perspective view of the present invention;

FIG. 2 is an exploded side view of the present invention;

FIG. 2A is an assembled side view of the present invention;

FIG. 3 is a cross-sectional view showing a menstrual cup and a collection bag of the present invention;

FIG. 4 is a schematic view of the present invention in a compressed status;

FIG. 5 is a schematic view of the present invention in use; and

FIG. 6 is a schematic view of the present invention collecting menstrual blood.

### DETAILED DESCRIPTION OF THE PREFERRED EMBODIMENT

Embodiments of the present invention will now be described, by way of example only, with reference to the accompanying drawings.

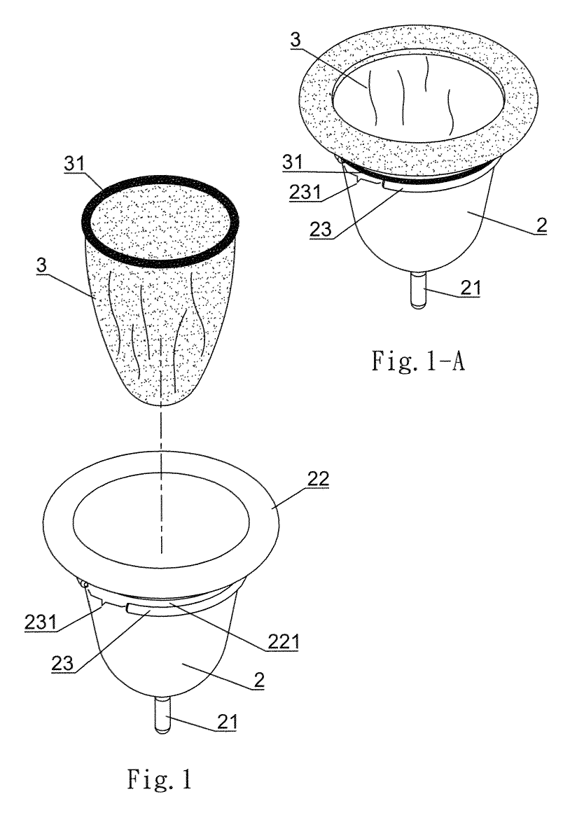

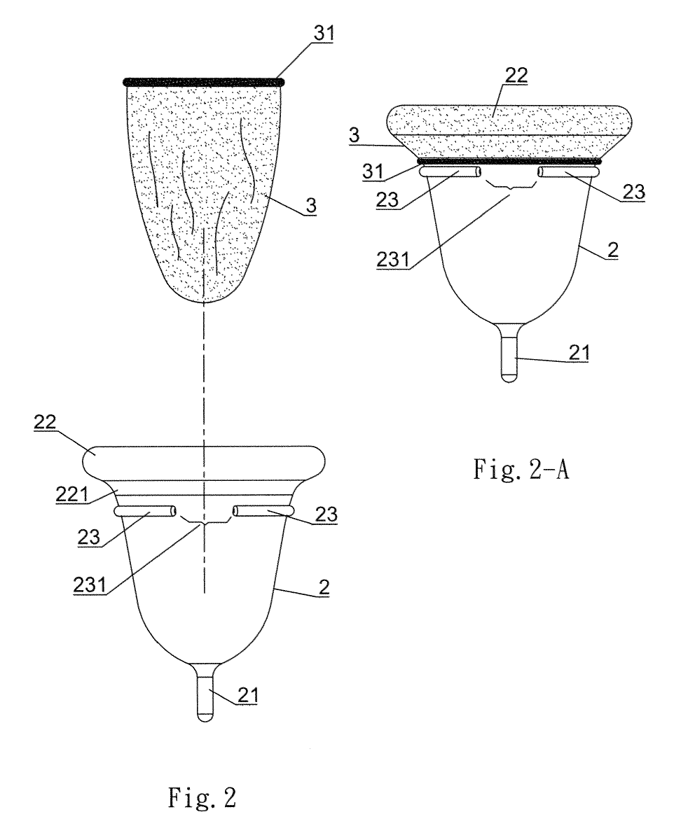

As shown in FIG. 1, FIGS. 1-A, FIG. 2, FIG. 2-A, a menstrual cup device of the present invention comprises a menstrual cup 2 and a collection bag 3.

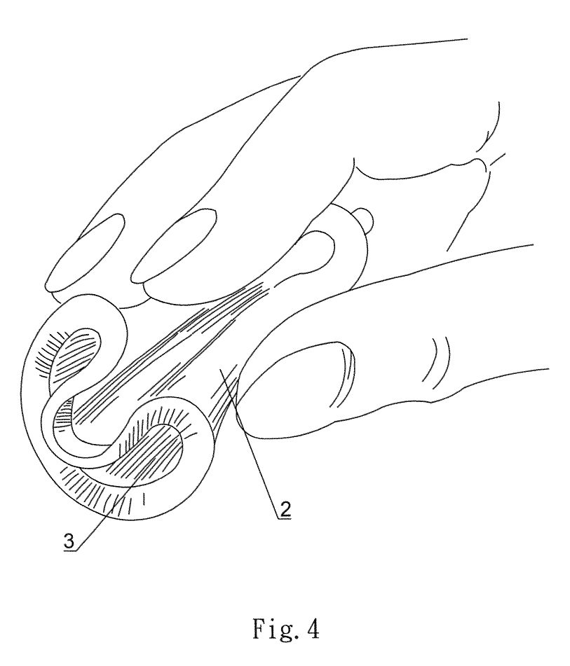

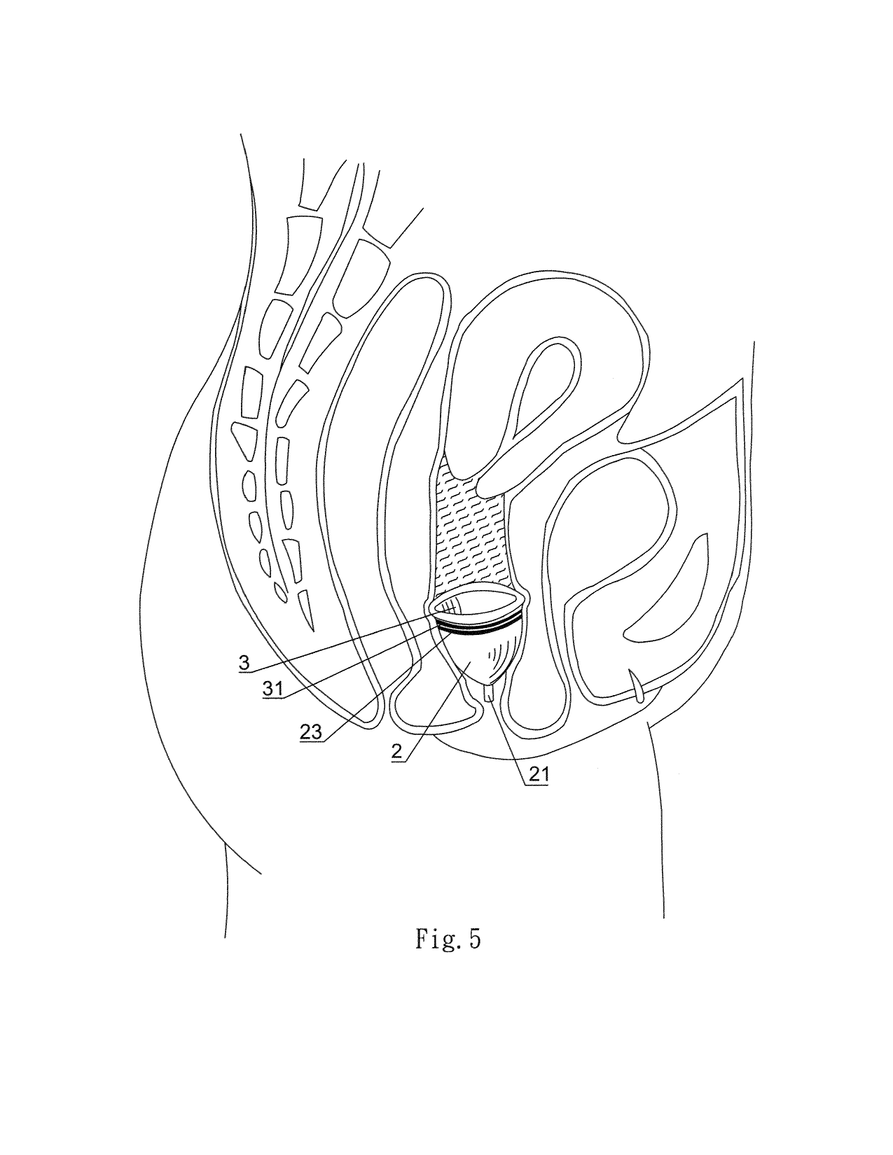

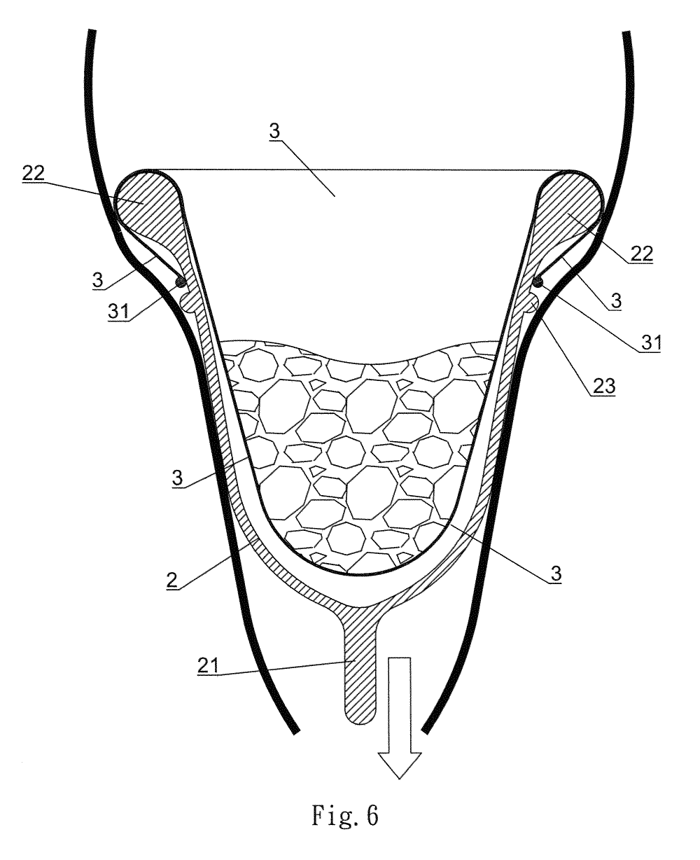

The menstrual cup 2 is made of expandable material, such as medical silica gel, so as to be compressed, as shown in FIG. 4. The menstrual cup 2 has resilience for restoring to its original shape after being compressed, as shown in FIGS. 5 and 6. The menstrual cup 2 comprises a pulling stem 21 at a lower end thereof, a circular lip 22 at an open upper end thereof, a circular groove 221 below the circular lip 22, and a circular rib 23 below the circular groove 221. The circular rib 23 is formed with at least one gap 231.

The collection bag 3 has a resilient restraining ring 31 at an open upper end thereof. The resilient restraining ring 31 is located in the circular groove 221 of the menstrual cup 2 after the collection bag 3 is fitted in the menstrual cup 2. The circular lip 22 is adapted to prevent the resilient restraining ring 31 from disengagement. The circular rib 23 is adapted to prevent the resilient restraining ring 31 from being pushed out of the circular groove 221 when the menstrual cup 2 is removed from the vagina. The collection bag 3 is disposed in the menstrual cup 2 securely when the menstrual cup 2 is compressed and inserted into the vagina or removed from the vagina. The collection bag 3 is disposable and replaceable. The present invention is convenient, safe, and hygienic.

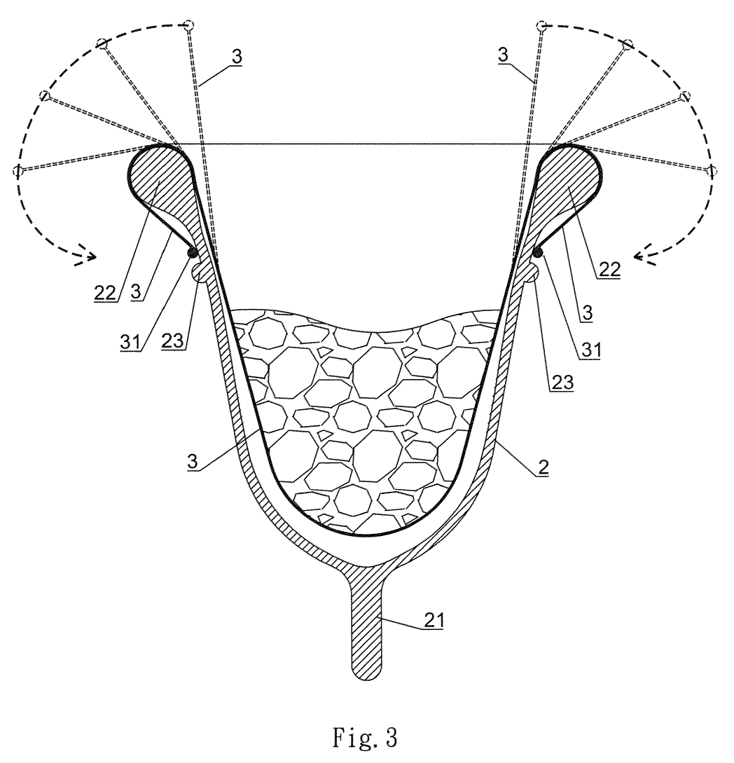

As shown in FIG. 1, FIGS. 1-A, FIG. 2, FIG. 2-A, FIGS. 3 through 6, a method of use of the present invention comprises the following steps of:
(a) expanding a resilient restraining ring 31 of a collection bag 3 to be located in a circular groove 221 of a menstrual cup for lowering the risk of disengagement;
(b) providing a circular rib 23 below the resilient restraining ring 31 fitted in the circular groove 221;
(c) placing the collection bag 3 in the menstrual cup 2 to become an integral one;
(d) compressing the menstrual cup 2 and the collection bag 3;
(e) inserting the compressed menstrual cup 2 and the collection bag 3 into the vagina, restoring the menstrual cup 2 to its original shape with the collection bag 3 wrapping a circular lip provided at an open upper end of the menstrual cup 2 for abutting against the vaginal canal;
(f) removing the menstrual cup 2 with the collection bag 3 from the vagina for replacement, with the circular rib 23 below the resilient restraining ring 31 to prevent the resilient restraining ring 31 form disengagement; and
(g) taking the collection bag 3 out via the resilient restraining ring 31 and replacing a new one.

As shown in FIG. 1, FIGS. 1-A, FIG. 2, and FIG. 2-A, the circular rib 23 located below the circular groove 221 of the menstrual cup 2 is formed with at least one gap 231 for a user to take the collection bag 3 out by poking his/her finger into the gap 231 when the resilient restraining ring 31 is constricted in the circular groove 221 which is located between the circular lip 22 of the menstrual cup 2 and the circular rib 23.

Although particular embodiments of the present invention have been described in detail for purposes of illustration, various modifications and enhancements may be made without departing from the spirit and scope of the present invention. Accordingly, the present invention is not to be limited except as by the appended claims.

What is claimed is:

1. A menstrual cup device, comprising:
a menstrual cup made of expandable material so as to be compressed, the menstrual cup having resilience for restoring to its original shape after being compressed, the menstrual cup comprising a pulling stem at a lower end thereof, a circular lip at an open upper end thereof, and a circular groove below the circular lip; and
a collection bag having a resilient restraining ring at an open upper end thereof;
thereby, the resilient restraining ring being expanded to be constricted in the circular groove of the menstrual cup, the circular lip being adapted to prevent the resilient restraining ring from disengagement, the menstrual cup with the collection bag being compressed and inserted into the vagina for collecting menstrual blood, the collection bag being disposable and replaceable.

2. The menstrual cup device as claimed in claimed 1, wherein the menstrual cup further comprises a circular rib below the circular groove for the resilient restraining ring to be constricted in the circular grove and located between the circular lip and the circular rib.

3. The menstrual cup device as claimed in claimed 1, wherein the menstrual cup further comprises a circular rib below the circular groove, the circular rib being formed with at least one gap for a user to take the collection bag out easily.

4. A method of use for applying a menstrual cup device, comprising the following steps of:
(a) expanding a resilient restraining ring of a collection bag to be located in a circular groove of a menstrual cup for lowering the risk of disengagement;
(b) providing a circular rib below the resilient restraining ring fitted in the circular groove;
(c) placing the collection bag in the menstrual cup to become an integral one;
(d) compressing the menstrual cup and the collection bag;
(e) inserting the compressed menstrual cup and the collection bag into the vagina, restoring the menstrual cup to its original shape with the collection bag wrapping a circular lip provided at an open upper end of the menstrual cup for abutting against the vaginal canal;
(f) removing the menstrual cup with the collection bag from the vagina for replacement, with the circular rib below the resilient restraining ring to prevent the resilient restraining ring form disengagement; and
(g) taking the collection bag out via the resilient restraining ring and replacing a new one.

5. The method of use for applying a menstrual cup device as claimed in claim 4, wherein the menstrual cup further comprises a circular rib below the circular groove, the circular rib being formed with at least one gap for a user to take the collection bag out easily.
